(19) United States
(12) Patent Application Publication
(10) Pub. No.: US 2015/0246836 A1
(43) Pub. Date: Sep. 3, 2015

(54) SOIL BALL FOR WATER PURIFICATION WITH INCREASED HARDNESS AND MICROBIAL POPULATION

(71) Applicant: Kyungpook National University Industry-Academic Cooperation Foundation, Daegu (KR)

(72) Inventors: Jae-Ho Shin, Daegu (KR); Gun-Seok Park, Gyeongsangnam-do (KR); Jin-Ho Shin, Daegu (KR); Jong-Hui Lim, Daegu (KR)

(21) Appl. No.: 14/347,015

(22) PCT Filed: Dec. 7, 2012
(86) PCT No.: PCT/KR12/10606

§ 371 Date: Mar. 25, 2014

(30) Foreign Application Priority Data
Nov. 6, 2012 (KR) 10-2012-0124910

(51) Int. Cl. C02F 3/34; C12N 1/16; C12N 1/20

(52) U.S. Cl. CPC C02F 3/348; C12N 1/20; C12N 1/16

(57) ABSTRACT

The present invention provides a soil ball for water purification with increased hardness and microbial population. According to the soil ball for water purification with increased hardness and microbial population of the present invention, the addition of a hardener increases the hardness such that the degradation of the soil ball in water is delayed, and the addition of a buffer solution prevents an increase in pH to increase the microbial population, thus improving the effect of water purification.

### TECHNICAL FIELD

The present invention relates to a soil ball for water purification with increased hardness and microbial population.

### BACKGROUND ART

Water pollution refers to a state in which the water quality is beyond the characteristics of natural water. That is, it refers to a phenomenon in which pollutants produced by human activities are introduced into surface water or ground water to cause deterioration of water quality and to limit the use of water resources or destroy the ecosystem. Water pollution sources are generally classified into point sources, where the discharge points of pollutants can be easily identified, and nonpoint or diffuse sources, where the discharge points of pollutants cannot be easily identified or where the pollution sources are diffuse and cause pollution. The point sources include living pollution sources, industrial wastewater, livestock wastewater, etc. The nonpoint sources are scattered over a large area by the discharge of pollutants due to precipitation and occur in the atmosphere and precipitation, mountainous areas, agricultural lands, metropolitan areas, etc. When water is polluted, organic substances, bacteria, etc. increase, and thus protozoa that feed on them also increase, and as a result, the photosynthetic plants are relatively reduced. While catfishes, carps, etc. can be found even in an area where the oxygen concentration is low, organisms other than bacteria and fungi cannot be found in an area where the pollution is serious.

In addition to the case where fishes and shells are directly affected by water pollution, people may be directly damaged by water pollution or indirectly damaged by other plants. The damages caused by toxic substances may be classified into acute and chronic types. While the acute damage can be easily determined, the chronic damage is gradually accumulated and appears slowly. Examples of the chronic damage include Minamata disease caused by mercury, Itai-Itai disease caused by cadmium (cd), etc., and the chronic damage caused by agricultural chemicals have recently been raised as a serious problem.

Water purification for solving the water pollution problems is carried out using various materials and technologies. Among others, according to ‘MANUFACTURING METHOD OF WATER PURIFICATION BALLS USING YELLOW EARTH AS MAIN RAW MATERIAL’ as a main component to prevent the occurrence of red tide, disclosed in Korean Patent No. 10-0301562, a loess ball is manufactured by mixing loess, elvan powder, and oak sawdust with water and forming pores in the middle thereof, followed by drying heating in a heating furnace, are released into water.

Currently used soil balls, which are manufactured by simply mixing effective microorganism activated solution, bokashi, and loess, are rapidly degraded in water, and thus they cannot properly serve as water purification. Moreover, the pH of these soil balls is acidic (pH 4.3) and thus has an adverse effect on the growth of microorganisms. However, there are not many studies on the soil balls for water purification to make up for these drawbacks.

### DISCLOSURE

### Technical Problem

The present inventors have studied methods for increasing the hardness of a soil ball for water purification and found that the addition of a hardener and a buffer solution to the soil ball for water purification increases the hardness of the soil ball and increases the microbial population, thus completing the present invention.

Accordingly, an object of the present invention is to provide a soil ball for water purification with increased hardness and microbial population.

### Technical Solution

The present invention provides a soil ball for water purification with increased hardness and microbial population.

### Advantageous Effects

According to the soil ball for water purification with increased hardness and microbial population of the present invention, the addition of a hardener increases the hardness such that the degradation of the soil ball in water is delayed, and the addition of a buffer solution prevents an increase in pH to increase the microbial population, thus improving the effect of water purification.

### DESCRIPTION OF DRAWINGS

FIG. 1 shows an image of a soil ball for water purification prepared in Example 1.

FIG. 2 shows the states of soil balls in Comparative Example 1 (FIG. 2A) and Example 2 (FIG. 2B) in flowing water tanks after 2 days.

FIG. 3 shows the states of soil balls in Comparative Example 1 (FIG. 3A) and Example 2 (FIG. 3B) in flowing water tanks after 7 days.

FIG. 4 shows the results of quantitative real time PCR analysis of microbial chromosomal DNA isolated from soil balls in Comparative Example 1 and Example 2.

FIGS. 5 and 6 show the results of microbial growth measured by metagenomics using the Personal Genome Machine (Ion Torrent) in metagenomic DNA isolated from soil balls in Comparative Example 1 and Example 2 for a detailed examination of microbial community.

### BEST MODE FOR CARRYING OUT THE INVENTION

The present invention provides a soil ball for water purification with increased hardness and microbial population, the soil ball comprising 80-90 wt % loess, 3-7 wt % effective microorganism activated solution (EMAS), 3-7 wt % bokashi, 0.5-2 wt % hardener, and 3-7% buffer solution.

The loess basically has water purification properties and is mixed with water to provide an appropriate viscosity and to bind other materials, thus defining the overall framework of the soil ball for water purification according to the present invention.

The effective microorganism activated solution is a liquid substance in which about 80 species of microorganisms, such as yeast, lactic acid bacteria, photosynthetic bacteria, actinomycetes, Bacillus spp, filamentous bacteria, etc., are fermented with rice bran or wheat bran for several days and has a deodorizing effect as well as water purification and antioxidative effects.

The bokashi is made by mixing the effective microorganism activated solution with fermentation microorganisms such as sawdust, rice bran, etc. and serves as an energy source for supplying nutrients to effective microorganisms mixed in the soil ball for water purification.

The hardener may be prepared by mixing calcium oxide and silicon dioxide in a molar ratio of 2-5:1, preferably 3:1, but not limited thereto.

The buffer solution may be a citric acid buffer solution of pH 4-4.5, but not limited thereto.

It is preferable that the soil ball for water purification is cultured in a culture chamber in a temperature range of 25 to 35° C. for 5 to 10 day, and this temperature range overlaps the activation temperature range of effective microorganisms. When the culture is normally completed, the effective microorganisms are grown to form hyphae across the entire surface of the soil ball for water purification, which are coated on the surface of the soil ball to be put into water, thus completing the cultured soil ball for water purification.

According to the soil ball for water purification with increased hardness and microbial population of the present invention, the addition of a hardener increases the hardness such that the degradation of the soil ball in water is delayed, and the addition of a buffer solution prevents an increase in pH to increase the microbial population, thus improving the effect of water purification.

### Mode for Invention

Hereinafter, preferable Examples are provided for better understanding of the present invention. However, the following Examples are provided only for illustrative purposes, and the present invention is not limited by the Examples.

### EXAMPLE 1

### Preparation of Soil Ball for Water Purification (0.5 wt % Hardener)

1-1. Preparation of Effective Microorganism (EM) Mixed Soil

500 g of EM mixed soil was prepared by adding 4.5 wt % water, 5 wt % EM activated solution, 5 wt % bokashi,

% hardener prepared by mixing calcium oxide and silicon dioxide in a molar ratio of 3:1, and 5 wt % citric acid buffer solution to 80 wt % loess and mixing the ingredients.

1-2. Culture of EM Mixed Soil

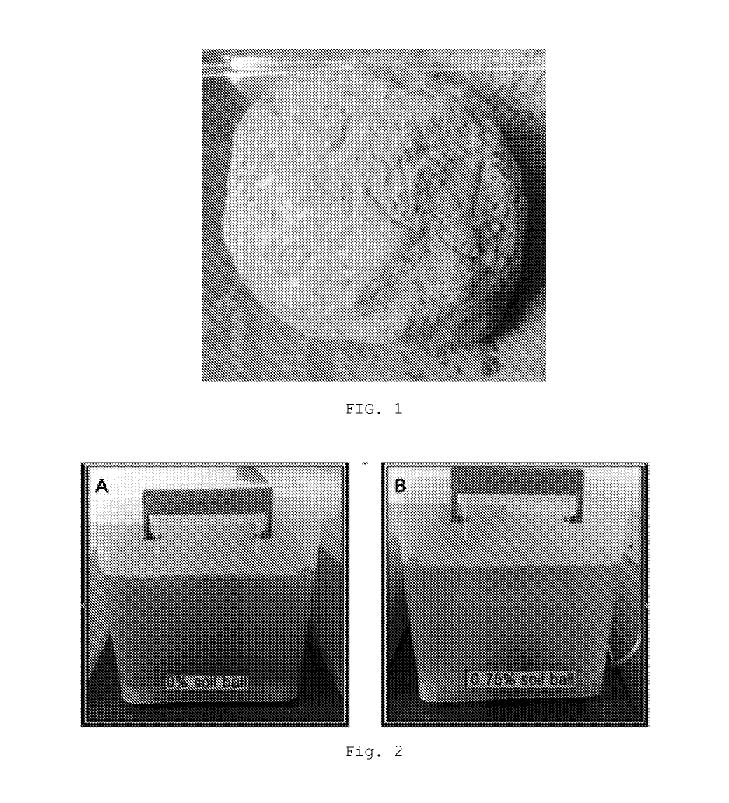

The EM mixed soil prepared in Example 1-1 was kept in a thermostatic chamber at 30° C. for 6 days to culture microorganisms, thus increasing the hardness of the soil ball and increasing the microbial population. An image of the completed soil ball for water purification is shown in FIG. 1.

As shown in FIG. 1, it was found that the surface of the EM mixed soil was coated with effective microorganisms and covered with white color.

### EXAMPLE 2

### Preparation of Soil Ball for Water Purification (0.75 wt % Hardener)

Soil ball for water purification was prepared by the same composition and method as in Example 1, except that 0.75 wt % hardener and 4.25 wt % water were used instead of 0.5 wt % hardener and 4.5 wt % water.

### EXAMPLE 3

### Preparation of Soil Ball for Water Purification (1.0 wt % Hardener)

Soil ball for water purification was prepared by the same composition and method as in Example 1, except that 1.0 wt % hardener and 4.0 wt % water were used instead of 0.5 wt % hardener and 4.5 wt % water.

### COMPARATIVE EXAMPLE 1

### Preparation of Soil Ball for Water Purification Containing No Hardener and Buffer Solution

Soil ball for water purification was prepared by the same composition and method as in Example 1, except that the hardener and buffer solution were not used and the remainder was filled with water.

### EXPERIMENTAL EXAMPLE 1

### Measurement of the Hardness of Soil Balls for Water Purification

1.1. Measurement of the Hardness of Soil Balls for Water Purification

The hardness of the soil balls for water purification prepared in Examples 1 to 3 and Comparative Example 1 was measured using a hardness tester with a 5 kg load. The results are shown in the following Table 1:

TABLE 1

| Soil balls for water purification | Hardness |
|---|---|
| Example 1 (0.5 wt % hardener) | 0.6 kg |
| Example 2 (0.75 wt % hardener) | 1.02 kg |
| Example 3 (1.0 wt % hardener) | 1.8 kg |
| Comparative Example 1 (0 wt % hardener) | 0.25 kg |

As shown in Table 1, it was found that when the amount of the hardener prepared by mixing calcium oxide and silicon dioxide in a molar ratio of 3:1 and added to the soil ball for water purification increased, the hardness of the soil ball also increased.

1-2. Measurement of the Degradation Rate of Soil Balls for Water Purification and Turbidity in Flowing Water Tanks

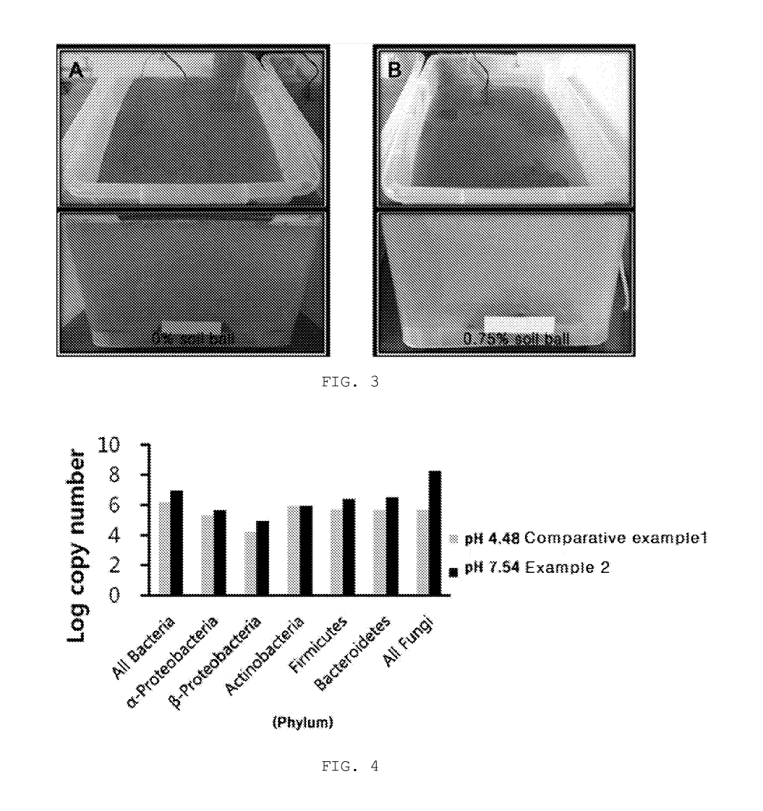

Each one of the soil balls (500 g) prepared in Example 2 (0.75 wt % hardener) and Comparative Example 1 (0 wt % hardener) was placed in a water tank in which 30 L water was contained and water flowed at a rate of 6 L/min, respectively, and the degradation rate was observed. The results are shown in FIGS. 2 and 3.

As shown in FIG. 2, as a result of observing the soil balls 2 days after being placed in the water tanks, the soil ball in Comparative Example 1 was completely degraded, while the soil ball in Example 2 started to degrade but maintained the overall shape.

As shown in FIG. 3, as a result of observing the soil balls 7 days after being placed in the water tanks, the soil ball in Comparative Example 1 was completely degraded and suspended, from which it can be seen that it cannot properly serve as a microbial carrier in actual rivers, while the soil ball in Example 2 was degraded but scattered in a small area, from which it can be seen that it still serves as a carrier.

Moreover, in order to determine the turbidity of water contained in a water tank, in which each of the soil balls in Example 2 and Comparative Example 1 was placed, at 0 day, 2 days, and 7 days, the absorbance was measured at 595 nm. The results are shown in the following Table 2:

TABLE 2

| | At 0 day | At 2 days | At 7 days |
|---|---|---|---|
| Containing only water | 0 | 0 | 0 |
| Comparative Example 1 | 0 | 1.314 | 1.411 |
| Example 2 | 0 | 0.257 | 0.293 |

As shown in Table 2, it was found that in the case of the soil ball in Comparative Example 1, the turbidity significantly increased when it was degraded, but in the case of the soil ball in Example 2, the turbidity was significantly lower than other soil balls, from which it is expected that the soil ball containing the hardener can properly serve as an effective microbial carrier.

### EXPERIMENTAL EXAMPLE 2

### Measurement of the pH of Soil Balls for Water Purification

The pH of the soil balls for water purification prepared in Examples 1 to 3 and Comparative Example 1 was measured. The results are shown in the following Table 3:

TABLE 3

| Soil balls for water purification | pH |
|---|---|
| Example 1 | 6.14 |
| Example 2 | 7.54 |
| Example 3 | 8.57 |
| Comparative Example 1 | 4.48 |

As shown in Table 3, the pH of the soil balls in Examples 1 to 3 was lower than pH 10 which is not suitable for microbial growth, from which it was found that the pH increased by the hardener was reduced by the citric acid buffer solution.

### EXPERIMENTAL EXAMPLE 3

### Measurement of the Degree of Microbial Growth in Soil Balls for Water Purification

In order to measure the degree of microbial growth that is changed by the addition of the hardener and the buffer solution, microbial chromosomal DNA was isolated from the soil balls in Comparative Example 1 and Example 2 and analyzed by quantitative real time PCR. The results are shown in FIG. 4.

As shown in FIG. 4, the population of effective microorganisms grown in the soil ball in Example 2 increased about 10 times, and the population of fungi that provide water purification and secrete plant growth hormones increased more than 100 times.

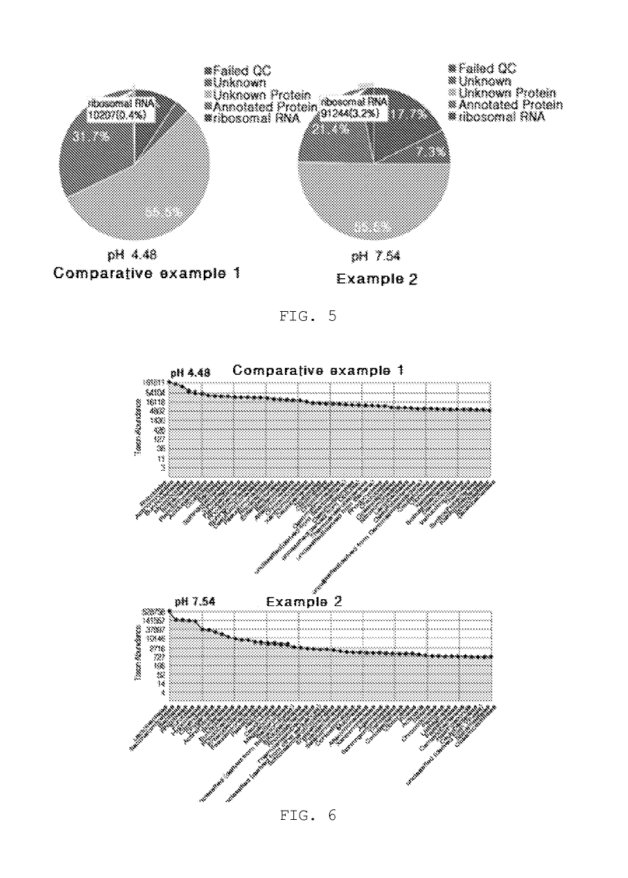

Moreover, for a detailed examination of microbial community in metagenomic DNA isolated from the soil balls in Comparative Example 1 and Example 2, the microbial growth was measured by metagenomics using the Personal Genome Machine (Ion Torrent). The results are shown in FIGS. 5 and 6.

As shown in FIG. 5, the number of rRNA sequences isolated from the soil ball in Comparative Example 1 where the pH was 4.48 was 10,207, while the number of rRNA sequences isolated from the soil ball in Example 2 where the pH was 7.54 was 91,244, which were higher about 9 times.

As shown in FIG. 6, as a result of examining the top 50 microbial communities based on the isolated rRNA sequences, the community of effective microorganisms such as Lactobacillales, Saccharomycetales, etc. was superior in the soil ball in Example 2, unlike the soil ball in Comparative Example 1.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

1. A soil ball for water purification with increased hardness and microbial population, the soil ball comprising 80-90 wt % loess, 3-7 wt % effective microorganism activated solution (EMAS), 3-7 wt % bokashi, 0.5-2 wt % hardener, and 3-7% buffer solution.

2. The soil ball of claim 1, wherein the hardener is prepared by mixing calcium oxide and silicon dioxide in a molar ratio of 2-5:1.

3. The soil ball of claim 1, wherein the buffer solution is a citric acid buffer solution of pH 4-4.5.

4. The soil ball of claim 1, wherein the surface of the soil ball for water purification is coated with effective microorganisms.
